(19) United States
(12) Patent Application Publication
(10) Pub. No.: US 2009/0197303 A1
(43) Pub. Date: Aug. 6, 2009

(54) PROCESS FOR PRODUCING DIPEPTIDES

(75) Inventors: Kuniki Kino, Chiba (JP); Atsushi Noguchi, Mie (JP); Yuji Nakazawa, Yamaguchi (JP); Makoto Yagasaki, Hofu-shi (JP)

Correspondence Address: FITZPATRICK CELLA HARPER & SCINTO, 30 ROCKEFELLER PLAZA, NEW YORK, NY, 10112, US

(73) Assignee: KYOWA HAKKO KOGYO CO., LTD., Chiyoda-ku, Tokyo (JP)

(21) Appl. No.: 12/097,806

(22) PCT Filed: Dec. 27, 2006
(86) PCT No.: PCT/JP2006/326024

§ 371 Date: Jun. 17, 2008

(30) Foreign Application Priority Data
Dec. 27, 2005 (JP) 2005-373874

(51) Int. Cl. C12P 21/02; C07K 16/18; C12N 15/11; C12N 15/00; C12N 1/21

(52) U.S. Cl. 435/69.1; 530/350; 536/23.1; 435/320.1; 435/252.33

(57) ABSTRACT

The present invention provides: a protein having dipeptide-synthesizing activity; DNA encoding the protein; a recombinant DNA comprising the DNA; a transformant transformed with the recombinant DNA; a process for producing the protein having dipeptide-synthesizing activity using the transformant or the like; a process for producing a dipeptide using the protein having dipeptide-synthesizing activity; and a process for producing a dipeptide using, as an enzyme source, a culture of a transformant or a microorganism which produces the protein having dipeptide-synthesizing activity or the like.

### TECHNICAL FIELD

The present invention relates to a process for producing a dipeptide.

### BACKGROUND ART

As for the method for large-scale peptide synthesis, chemical synthesis methods (liquid phase method and solid phase method), enzymatic synthesis methods and biological synthesis methods utilizing recombinant DNA techniques are known. Currently, the enzymatic synthesis methods and biological synthesis methods are mainly employed for the synthesis of long-chain peptides longer than dozens of residues, and the chemical synthesis methods and enzymatic synthesis methods are mainly employed for the synthesis of short-chain peptides of two to several residues.

In the synthesis of short-chain peptides by the chemical synthesis methods, operations such as introduction and removal of protective groups for functional groups are necessary, and racemates are also formed as by-products. The chemical synthesis methods are thus considered to be disadvantageous in respect of cost and efficiency.

As to the synthesis of short-chain peptides by the enzymatic methods, the following methods are known: a method utilizing reverse reaction of protease (see non-patent document No. 1); methods utilizing transesterifying enzymes (patent document Nos. 1 to 3 and non-patent document No. 2); methods utilizing thermostable aminoacyl t-RNA synthetase (patent document Nos. 4 to 7); and methods utilizing non-ribosomal peptide synthetase (hereinafter referred to as NRPS) (see non-patent document Nos. 3 and 4 and patent document Nos. 8 and 9).

However, the method utilizing reverse reaction of protease requires introduction and removal of protective groups for functional groups of amino acids used as substrates, which causes difficulties in raising the efficiency of peptide-forming reaction and in preventing peptidolytic reaction. The methods utilizing transesterifying enzymes, which require esterification of amino acids used as substrates, have problems such as inefficiency and decrease of yields due to decomposition of amino acid esters as substrates and the formed peptides. The methods utilizing thermostable aminoacyl t-RNA synthetase have the defects that the expression of the enzyme and the prevention of side reactions forming by-products other than the desired products are difficult. The methods utilizing NRPS are inefficient in that the expression of the enzyme by recombinant DNA techniques is difficult because the enzyme molecule is huge, and in that the supply of coenzyme 4′-phosphopantetheine is necessary.

On the other hand, there exist a group of peptide synthetases that have enzyme molecular weight lower than that of NRPS and do not require coenzyme 4′-phosphopantetheine; for example, γ-glutamylcysteine synthetase, D-alanyl-D-alanine (D-Ala-D-Ala) ligase and poly-γ-glutamate synthetase. Most of these enzymes utilize D-amino acids as substrates or catalyze peptide bond formation at the γ-carboxyl group. Because of such properties, they can not be used for the synthesis of short-chain peptides by peptide bond formation at the α-carboxyl group of L-amino acid.

The only known example of an enzyme having the activity to catalyze the formation of a peptide bond at the α-carboxyl group of L-amino acid to form a dipeptide is bacilysin (dipeptide antibiotic derived from a microorganism belonging to the genus Bacillus) synthetase. Bacilysin synthetase is known to have the activity to synthesize bacilysin [L-alanyl-L-anticapsin (L-Ala-L-anticapsin)] and L-alanyl-L-alanine (L-Ala-L-Ala) (see non-patent document Nos. 5 and 6). Recently, it has been reported that this enzyme has the activity to form various kinds of dipeptides from various combinations of the same or different free amino acids (see patent document No. 10 and non-patent document No. 7).

However, there exists a need for a novel dipeptide-synthesizing enzyme which has substrate specificity different from that of the above enzyme, because the above enzyme can not form all dipeptides efficiently due to its substrate specificity.

The nucleotide sequences of the chromosomal DNAs and the presumed nucleotide sequences of genes of Bacillus licheniformis ATCC 14580 and Bacillus licheniformis DSM13 are both known (see non-patent document No. 8). However, it is not known whether BL00235 gene and BLi04240 gene in the above genes are actually genes encoding proteins having a function, not to mention the function of proteins encoded by BL00235 gene and BLi04240 gene.

Patent document No. 1:
WO03/010187 pamphlet
   Patent document No. 2:
WO03/010307 pamphlet
   Patent document No. 3:
WO03/010189 pamphlet
   Patent document No. 4:
Japanese Published Unexamined Patent Application No. 146539/83
   Patent document No. 5:
Japanese Published Unexamined Patent Application No. 209991/83
   Patent document No. 6:
Japanese Published Unexamined Patent Application No. 209992/83
   Patent document No. 7:
Japanese Published Unexamined Patent Application No. 106298/84
   Patent document No. 8:
U.S. Pat. No. 5,795,738
   Patent document No. 9:
U.S. Pat. No. 5,652,116
   Patent document No. 10:
WO04/058960 pamphlet
   Non-patent document No. 1:
J. Biol. Chem., 119, 707-720 (1937)
   Non-patent document No. 2:
J. Biotechnol., 115, 211-220 (2005)
   Non-patent document No. 3:
Chem. Biol., 7, 373-384 (2000)
   Non-patent document No. 4:
FEBS Lett., 498, 42-45 (2001)
   Non-patent document No. 5:
J. Ind. Microbiol., 2, 201-208 (1987)
   Non-patent document No. 6:
Enzyme Microb. Technol., 29, 400-406 (2001)
   Non-patent document No. 7:
J. Bacteriol., 187, 5195-5202 (2005)
   Non-patent document No. 8:
http://gib.genes.nig.ac.jp/single/index.php?spid=Blic_DSM13_NOVOZYMES

### DISCLOSURE OF THE INVENTION

### Problems to be Solved by the Invention

An object of the present invention is to provide: a protein having dipeptide-synthesizing activity; DNA encoding the protein; a recombinant DNA comprising the DNA; a transformant transformed with the recombinant DNA; a process for producing the protein having dipeptide-synthesizing activity using the transformant or the like; a process for producing a dipeptide using the protein having dipeptide-synthesizing activity; and a process for producing a dipeptide using, as an enzyme source, a culture of a transformant or a microorganism which produces the protein having dipeptide-synthesizing activity or the like.

### Means for Solving the Problems

The present invention relates to the following (1) to (10).
(1) A protein according to any of the following [1] to

[3]:
[1] a protein having the amino acid sequence of SEQ ID NO: 1;
[2] a protein consisting of an amino acid sequence wherein one or more amino acid residues are deleted, substituted or added in the amino acid sequence of SEQ ID NO: 1 and having dipeptide-synthesizing activity; and

[3] a protein consisting of an amino acid sequence which has 80% or more homology to the amino acid sequence of SEQ ID NO: 1 and having dipeptide-synthesizing activity.
(2) A DNA according to any of the following [1] to [3]:
   [1] DNA encoding the protein according to the above (1);
   [2] DNA having the nucleotide sequence of SEQ ID NO: 2; and
   [3] DNA which hybridizes with DNA having a nucleotide sequence complementary to the nucleotide sequence of SEQ ID NO: 2 under stringent conditions and which encodes a protein having dipeptide-synthesizing activity.
(3) A recombinant DNA comprising the DNA according to the above (2).
(4) A transformant carrying the recombinant DNA according to the above (3).
(5) The transformant according to the above (4), wherein the transformant is a transformant obtained by using a microorganism as a host.
(6) The transformant according to the above (5), wherein the microorganism is a microorganism belonging to the genus Escherichia.
(7) A process for producing the protein according to the above (1), which comprises culturing a microorganism having the ability to produce the protein according to the above (1) in a medium, allowing the protein to form and accumulate in the culture, and recovering the protein from the culture.
(8) The process according to the above (7), wherein the microorganism having the ability to produce the protein according to the above (1) is the transformant according to any one of the above (4) to (6).
(9) A process for producing a dipeptide which comprises allowing a culture of a microorganism having the ability to produce the protein according to the above (1) or a treated culture, or the protein according to the above (1), and one or more kinds of amino acids to be present in an aqueous medium, allowing the dipeptide to form and accumulate in the medium, and recovering the dipeptide from the medium.
(10) The process according to the above (9), wherein the microorganism having the ability to produce the protein according to the above (1) is the transformant according to any one of the above (4) to (6).

### EFFECT OF THE INVENTION

In accordance with the present invention, a protein having the activity to synthesize a dipeptide can be produced, and a dipeptide can be produced by using the protein, or a transformant or a microorganism which has the ability to produce the protein.

### BRIEF DESCRIPTION OF THE DRAWINGS

FIG. 1 shows the steps for constructing plasmid pBL00235.

### EXPLANATION OF SYMBOLS

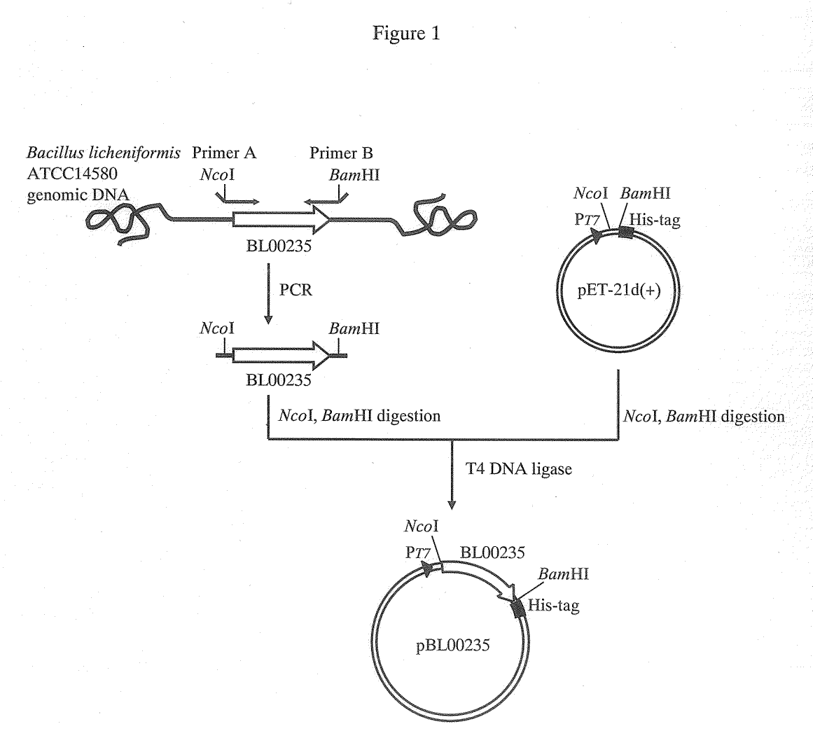

In FIG. 1, BL00235 represents BL00235 gene derived from Bacillus licheniformis ATCC 14580, PT7 represents T7 promoter gene, and His-tag represents the histidine-tag (His-tag) sequence.

### BEST MODES FOR CARRYING OUT THE INVENTION

### 1. Proteins of the Present Invention

The proteins of the present invention include:

[1] a protein having the amino acid sequence of SEQ ID NO: 1,
[2] a protein consisting of an amino acid sequence wherein one or more amino acid residues are deleted, substituted or added in the amino acid sequence of SEQ ID NO: 1 and having dipeptide-synthesizing activity; and
[3] a protein consisting of an amino acid sequence which has 80% or more homology to the amino acid sequence of SEQ ID NO: 1 and having dipeptide-synthesizing activity.

In the present specification, “dipeptide-synthesizing activity” refers to the activity to form a peptide bond between two amino acids, preferably the activity to form a peptide bond between a carboxyl group at the α position of an amino acid and an amino group of another amino acid.

The above protein consisting of an amino acid sequence wherein one or more amino acid residues are deleted, substituted or added and having dipeptide-synthesizing activity can be obtained, for example, by introducing a site-directed mutation into DNA encoding a protein consisting of the amino acid sequence of SEQ ID NO: 1 by site-directed mutagenesis described in Molecular Cloning, A Laboratory Manual, Third Edition, Cold Spring Harbor Laboratory Press (2001) (hereinafter referred to as Molecular Cloning, Third Edition); Current Protocols in Molecular Biology, John Wiley & Sons (1987-1997) (hereinafter referred to as Current Protocols in Molecular Biology); Nucleic Acids Research, 10, 6487 (1982); Proc. Natl. Acad. Sci. USA, 79, 6409 (1982); Gene, 34, 315 (1985); Nucleic Acids Research, 13, 4431 (1985); Proc. Natl. Acad. Sci. USA, 82, 488 (1985), etc.

The number of amino acid residues which are deleted, substituted or added is not specifically limited, but is within the range where deletion, substitution or addition is possible by known methods such as the above site-directed mutagenesis. The suitable number is 1 to dozens, preferably 1 to 20, more preferably 1 to 10, further preferably 1 to 5.

The expression “one or more amino acid residues are deleted, substituted or added in the amino acid sequence of SEQ ID NO: 1” means that the amino acid sequence may contain deletion, substitution or addition of a single or plural amino acid residues at an arbitrary position therein.

Deletion or addition of amino acid residues may be contained, for example, in the N-terminal or C-terminal one to several amino acid region of the amino acid sequence of SEQ ID NO: 1.

Deletion, substitution and addition may be simultaneously contained in one sequence, and amino acids to be substituted or added may be either natural or not. Examples of the natural amino acids are L-alanine, L-arginine, L-asparagine, L-aspartic acid, L-glutamine, L-glutamic acid, glycine, L-histidine, L-isoleucine, L-leucine, L-lysine, L-methionine, L-phenylalanine, L-proline, L-serine, L-threonine, L-tryptophan, L-tyrosine, L-valine and L-cysteine.

The following are examples of the amino acids capable of mutual substitution. The amino acids in the same group can be mutually substituted.
Group A: leucine, isoleucine, norleucine, valine, norvaline, alanine, 2-aminobutanoic acid, methionine, O-methylserine, t-butylglycine, t-butylalanine, cyclohexylalanine
Group B: aspartic acid, glutamic acid, isoaspartic acid, isoglutamic acid, 2-aminoadipic acid, 2-aminosuberic acid
Group C: asparagine, glutamine
Group D: lysine, arginine, ornithine, 2,4-diaminobutanoic acid, 2,3-diaminopropionic acid
Group E: proline, 3-hydroxyproline, 4-hydroxyproline
Group F: serine, threonine, homoserine
Group G: phenylalanine, tyrosine

In order that the protein of the present invention may have dipeptide-synthesizing activity, it is desirable that the homology of its amino acid sequence to the amino acid sequence of SEQ ID NO: 1 is 80% or more, preferably 90% or more, more preferably 95% or more, further preferably 98% or more, and particularly preferably 99% or more.

The homology among amino acid sequences and nucleotide sequences can be determined by using algorithm BLAST by Karlin and Altschul [Pro. Natl. Acad. Sci. USA, 90, 5873 (1993)] and FASTA [Methods Enzymol., 183, 63 (1990)]. On the basis of the algorithm BLAST, programs such as BLASTN and BLASTX have been developed [J. Mol. Biol., 215, 403 (1990)]. When a nucleotide sequence is analyzed by BLASTN on the basis of BLAST, the parameters, for instance, are as follows: score=100 and wordlength=12. When an amino acid sequence is analyzed by BLASTX on the basis of BLAST, the parameters, for instance, are as follows: score=50 and wordlength=3. When BLAST and Gapped BLAST programs are used, default parameters of each program are used. The specific techniques for these analyses are known (http://www.ncbi.nlm.nih.gov.).

It is possible to confirm that the protein of the present invention is a protein having dipeptide-synthesizing activity, for example, in the following manner. That is, a transformant expressing the protein of the present invention is prepared by recombinant DNA techniques, the protein of the present invention is produced using the transformant, and then the protein of the present invention, one or more kinds of amino acids, preferably two kinds of amino acids selected from the group consisting of L-amino acids and glycine, and ATP are allowed to be present in an aqueous medium, followed by HPLC analysis or the like to know whether a dipeptide is formed and accumulated in the aqueous medium.

### 2. DNAs of the Present Invention

The DNAs of the present invention include:

[1] DNA encoding the protein of the present invention according to [1] to [3] in the above 1;
[2] DNA having the nucleotide sequence of SEQ ID NO: 2; and
[3] DNA which hybridizes with DNA having a nucleotide sequence complementary to the nucleotide sequence of SEQ ID NO: 2 under stringent conditions and which encodes a protein having dipeptide-synthesizing activity.

“To hybridize” refers to a step of hybridization of DNA with DNA having a specific nucleotide sequence or a part of the DNA. Therefore, the nucleotide sequence of the DNA having a specific nucleotide sequence or a part of the DNA may be DNA which is long enough to be useful as a probe for Northern or Southern blot analysis or to be used as an oligonucleotide primer for PCR analysis. DNAs used as a probe include DNAs consisting of 100 or more nucleotides, preferably 200 or more nucleotides, more preferably 500 or more nucleotides, and DNAs used as a primer include DNAs consisting of 17 or more nucleotides, preferably 20 or more nucleotides, more preferably 25 or more nucleotides.

The method for hybridization of DNA is well known. The conditions for hybridization can be determined and hybridization experiments can be carried out, for example, according to the methods described in Molecular Cloning, Third Edition (2001); Methods for General and Molecular Bacteriology, ASM Press (1994); Immunology methods manual, Academic press (Molecular), and many other standard textbooks.

Hybridization under the above stringent conditions is carried out, for example, as follows. A filter with DNA immobilized thereon and a probe DNA are incubated in a solution comprising 50% formamide, 5×SSC (750 mM sodium chloride and 75 mM sodium citrate), 50 mM sodium phosphate (pH 7.6), 5×Denhardt's solution, 10% dextran sulfate and 20 μg/l denatured salmon sperm DNA at 42° C. overnight, and after the incubation, the filter is washed in 0.2×SSC solution (ca. 65° C.). The stringent conditions may be adjusted according to the length of a chain of a probe DNA and the GC content and can be set up by the method described in Molecular Cloning, Third Edition, etc. Less stringent conditions can also be employed. Modification of the stringent conditions can be made by adjusting the concentration of formamide (the conditions become less stringent as the concentration of formamide is lowered) and by changing the salt concentrations and the temperature conditions. Hybridization under less stringent conditions is carried out, for example, by incubating a filter with DNA immobilized thereon and a probe DNA in a solution comprising 6×SSCE (20×SSCE: 3 mol/l sodium chloride, 0.2 mol/l sodium dihydrogenphosphate and 0.02 mol/l EDTA, pH 7.4), 0.5% SDS, 30% formamide and 100 μg/l denatured salmon sperm DNA at 37° C. overnight, and washing the filter with 1×SSC solution containing 0.1% SDS (50° C.). Hybridization under still less stringent conditions is carried out by hybridization under the above less stringent conditions followed by washing using a solution having a high salt concentration (for example, 5×SSC).

Various conditions described above can also be established by adding a blocking reagent used to reduce the background of hybridization or changing the reagent. The addition of the above blocking reagent may be accompanied by changes of conditions for hybridization to make the conditions suitable for the purpose.

The above DNA capable of hybridization under stringent conditions includes DNA having at least 80% homology, preferably 90% or more homology, more preferably 95% or more homology, further preferably 98% or more homology, particularly preferably 99% or more homology to the nucleotide sequence of SEQ ID NO: 2 as calculated by use of programs such as BLAST and FASTA described above based on the above parameters.

The homology among nucleotide sequences can be determined by using programs such as BLAST and FASTA described above.

It is possible to confirm that the DNA hybridizing with the above DNA under stringent conditions is DNA encoding a protein having dipeptide-synthesizing activity in the following manner. That is, a recombinant DNA expressing the DNA is prepared and a protein is purified from the culture obtained by culturing a microorganism obtained by introducing the recombinant DNA into a host cell. Then, the purified protein as an enzyme source and one or more kinds of amino acids, preferably two kinds of amino acids selected from the group consisting of L-amino acids and glycine are allowed to be present in an aqueous medium, followed by HPLC analysis or the like to know whether a dipeptide is formed and accumulated in the aqueous medium.

### 3. Microorganisms and Transformants Used in the Production Process of the Present Invention

There is not any specific restriction as to the microorganisms and transformants used in the production process of the present invention, so long as they are microorganisms and transformants having the ability to produce the protein of the present invention. Suitable examples of the microorganisms include those belonging to the genus Bacillus, preferably those belonging to Bacillus licheniformis, more preferably Bacillus licheniformis ATCC 14580 and Bacillus licheniformis DSM13. Suitable examples of the transformants include those transformed with DNA encoding the protein of the present invention. Bacillus licheniformis ATCC 14580 can be obtained from American Type Culture Collection, which is a bioresource center in U.S.A., and Bacillus licheniformis DSM13 can be obtained from Deutsche Sammlung von Mikroorganismen und Zellkulturen GmbH, which is a bioresource preservation institute in Germany.

Examples of the transformants transformed with DNA encoding the protein of the present invention are those obtained by transforming a host cell by a known method using a recombinant DNA comprising the DNA of the above 2. Examples of the host cells include procaryotes such as bacterial cells, yeast cells, animal cells, insect cells and plant cells, preferably procaryotes such as bacterial cells, more preferably bacteria, further preferably microorganisms belonging to the genus Escherichia.

### 4. Preparation of the DNA of the Present Invention

The DNA of the present invention can be obtained, for example, by Southern hybridization of a chromosomal DNA library from a microorganism belonging to the genus Bacillus, preferably a microorganism belonging to Bacillus licheniformis, more preferably Bacillus licheniformis ATCC 14580 or Bacillus licheniformis DSM13, using a probe designed based on the nucleotide sequence of SEQ ID NO: 2, or by PCR [PCR Protocols, Academic Press (1990)] using primer DNAs designed based on the nucleotide sequence of SEQ ID NO: 2, and as a template, the chromosomal DNA of a microorganism, preferably a microorganism belonging to the genus Bacillus, more preferably a microorganism belonging to Bacillus licheniformis, further preferably Bacillus licheniformis ATCC 14580 or Bacillus licheniformis DSM13.

The DNA of the present invention or DNA used in the production process of the present invention can also be obtained by conducting a search through various gene sequence databases for a sequence having 80% or more homology, preferably 90% or more homology, more preferably 95% or more homology, further preferably 98% or more homology, particularly preferably 99% or more homology to the nucleotide sequence of DNA encoding the amino acid sequence of SEQ ID NO: 1, and obtaining the desired DNA, based on the nucleotide sequence obtained by the search, from a chromosomal DNA or cDNA library of an organism having the nucleotide sequence according to the above-described method.

The obtained DNA, as such or after cleavage with appropriate restriction enzymes, is inserted into a vector by a conventional method, and the obtained recombinant DNA is introduced into a host cell. Then, the nucleotide sequence of the DNA can be determined by a conventional sequencing method such as the dideoxy method [Proc. Natl. Acad. Sci., USA, 74, 5463 (1977)] or by using a nucleotide sequencer such as Applied Biosystems 3700 DNA Analyzer (Applied Biosystems).

In cases where the obtained DNA is found to be a partial DNA by the analysis of nucleotide sequence, the full length DNA can be obtained by Southern hybridization of a chromosomal DNA library using the partial DNA as a probe.

It is also possible to prepare the desired DNA by chemical synthesis using a DNA synthesizer (e.g., Model 8905, PerSeptive Biosystems) based on the determined nucleotide sequence of the DNA.

An example of the DNA that can be obtained by the above-described method is DNA having the nucleotide sequence of SEQ ID NO: 2.

Examples of the vectors for inserting the DNA of the present invention include pBluescript II KS(+) (Stratagene), pDIRECT [Nucleic Acids Res., 18, 6069 (1990)], pCR-Script Amp SK(+) (Stratagene), pT7Blue (Novagen, Inc.), pCR II (Invitrogen Corp.) and pCR-TRAP (Genhunter Corp.).

As the host cell, microorganisms belonging to the genus Escherichia, etc. can be used. Examples of the microorganisms belonging to the genus Escherichia include Escherichia coli XL1-Blue, Escherichia coli XL2-Blue, Escherichia coli DH1, Escherichia coli MC1000, Escherichia coli ATCC 12435, Escherichia coli W1485, Escherichia coli JM109, Escherichia coli HB101, Escherichia coli No. 49, Escherichia coli W3110, Escherichia coli NY49, Escherichia coli MP347, Escherichia coli NM522, Escherichia coli BL21 and Escherichia coli ME8415.

Introduction of the recombinant DNA can be carried out by any of the methods for introducing DNA into the above host cells, for example, the method using calcium ion [Proc. Natl. Acad. Sci. USA, 69, 2110 (1972)], the protoplast method (Japanese Published Unexamined Patent Application No. 248394/88) and electroporation [Nucleic Acids Res., 16, 6127 (1988)].

An example of the transformant of the present invention obtained by the above method is Escherichia coli BL21/pBL00235, which is a microorganism carrying a recombinant DNA comprising DNA having the sequence of SEQ ID NO: 2.

### 5. Process for Producing the Transformant and the Microorganism Used in the Production Process of the Present Invention

On the basis of the DNA of the present invention, a DNA fragment of an appropriate length comprising a region encoding the protein of the present invention is prepared according to need. A transformant having enhanced productivity of the protein can be obtained by replacing a nucleotide in the nucleotide sequence of the region encoding the protein so as to make a codon most suitable for the expression in a host cell.

The DNA fragment is inserted downstream of a promoter in an appropriate expression vector to prepare a recombinant DNA.

A transformant which produces the protein of the present invention can be obtained by introducing the recombinant DNA into a host cell suited for the expression vector.

As the host cell, any bacterial cells, yeast cells, animal cells, insect cells, plant cells, etc. that are capable of expressing the desired gene can be used.

The expression vectors that can be employed are those capable of autonomous replication or integration into the chromosome in the above host cells and comprising a promoter at a position appropriate for the transcription of the DNA of the present invention.

When a procaryote such as a bacterium is used as the host cell, it is preferred that the recombinant DNA comprising the DNA of the present invention is a recombinant DNA which is capable of autonomous replication in the procaryote and which comprises a promoter, a ribosome binding sequence, the DNA of the present invention and a transcription termination sequence. The recombinant DNA may further comprise a gene regulating the promoter.

Examples of suitable expression vectors are pBTrp2, pBTac1 and pBTac2 (products of Boehringer Mannheim GmbH), pHelix1 (Roche Diagnostics Corp.), pKK233-2 (Amersham Pharmacia Biotech), pSE280 (Invitrogen Corp.), pGEMEX-1 (Promega Corp.), pQE-8 (Qiagen, Inc.), pET-3 (Novagen, Inc.), pKYP10 (Japanese Published Unexamined Patent Application No. 110600/83), pKYP200 [Agric. Biol. Chem., 48, 669 (1984)], pLSA1 [Agric. Biol. Chem., 53, 277 (1989)], pGEL1 [Proc. Natl. Acad. Sci. USA, 82, 4306 (1985)], pBluescript II SK(+), pBluescript II KS(−) (Stratagene), pTrS30 [prepared from Escherichia coli JM109/pTrS30 (FERM BP-5407)], pTrS32 [prepared from Escherichia coli JM109/pTrS32 (FERM BP-5408)], pPAC31 (WO98/12343), pUC19 [Gene, 33, 103 (1985)], pSTV28 (Takara Shuzo Co., Ltd.), pUC118 (Takara Shuzo Co., Ltd.) and pPA1 (Japanese Published Unexamined Patent Application No. 233798/88).

As the promoter, any promoters capable of functioning in host cells such as Escherichia coli can be used. For example, promoters derived from Escherichia coli or phage, such as trp promoter (Pₜᵣₚ), lac promoter (P_{lac}), P_{L} promoter, P_{R} promoter and P_{SE} promoter, SPO1 promoter, SPO2 promoter and penP promoter can be used. Artificially designed and modified promoters such as a promoter in which two Pₜᵣₚs are combined in tandem, tac promoter, lacT7 promoter and letI promoter, etc. can also be used.

Also useful are promoters such as xylA promoter for the expression in microorganisms belonging to the genus Bacillus [Appl. Microbiol. Biotechnol., 35, 594-599 (1991)] and P54-6 promoter for the expression in microorganisms belonging to the genus Corynebacterium [Appl. Microbiol. Biotechnol., 53, 674-679 (2000)].

It is preferred to use a plasmid in which the distance between the Shine-Dalgarno sequence (ribosome binding sequence) and the initiation codon is adjusted to an appropriate length (e.g., 6 to 18 nucleotides).

In the recombinant DNA wherein the DNA of the present invention is ligated to an expression vector, the transcription termination sequence is not essential, but it is preferred to place the transcription termination sequence immediately downstream of the structural gene.

An example of such recombinant DNA is pBL00235.

Examples of procaryotes include microorganisms belonging to the genera Escherichia, Serratia, Bacillus, Brevibacterium, Corynebacterium, Microbacterium, Pseudomonas, Agrobacterium, Alicyclobacillus, Anabaena, Anacystis, Arthrobacter, Azotobacter, Chromatium, Erwinia, Methylobacterium, Phormidium, Rhodobacter, Rhodopseudomonas, Rhodospirillum, Scenedesmus, Streptomyces, Synechoccus and Zymomonas. Specific examples are Escherichia coli XL1-Blue, Escherichia coli XL2-Blue, Escherichia coli DH1, Escherichia coli DH5a, Escherichia coli MC1000, Escherichia coli KY3276, Escherichia coli W1485, Escherichia coli JM109, Escherichia coli HB101, Escherichia coli No. 49, Escherichia coli W3110, Escherichia coli NY49, Escherichia coli MP347, Escherichia coli NM522, Escherichia coli BL21, Bacillus subtilis ATCC 33712, Bacillus megaterium, Brevibacterium ammoniagenes, Brevibacterium immariophilum ATCC 14068, Brevibacterium saccharolyticum ATCC 14066, Brevibacterium flavum ATCC 14067, Brevibacterium lactofermentum ATCC 13869, Corynebacterium glutamicum ATCC 13032, Corynebacterium glutamicum ATCC 14297, Corynebacterium acetoacidophilum ATCC 13870, Microbacterium ammoniaphilum ATCC 15354, Serratia ficaria, Serratia fonticola, Serratia liquefaciens, Serratia marcescens, Pseudomonas sp. D-0110, Agrobacterium radiobacter, Agrobacterium rhizogenes, Agrobacterium rubi, Anabaena cylindrica, Anabaena doliolum, Anabaena flos-aquae, Arthrobacter aurescens, Arthrobacter citreus, Arthrobacter globiformis, Arthrobacter hydrocarboglutamicus, Arthrobacter mysorens, Arthrobacter nicotianae, Arthrobacter paraffineus, Arthrobacter protophormiae, Arthrobacter roseoparaffinus, Arthrobacter sulfureus, Arthrobacter ureafaciens, Chromatium buderi, Chromatium tepidum, Chromatium vinosum, Chromatium warmingii, Chromatium fluviatile, Erwinia uredovora, Erwinia carotovora, Erwinia ananas, Erwinia herbicola, Erwinia punctata, Erwinia terreus, Methylobacterium rhodesianum, Methylobacterium extorquens, Phormidium sp. ATCC 29409, Rhodobacter capsulatus, Rhodobacter sphaeroides, Rhodopseudomonas blastica, Rhodopseudomonas marina, Rhodopseudomonas palustris, Rhodospirillum rubrum, Rhodospirillum salexigens, Rhodospirillum salinarum, Streptomyces ambofaciens, Streptomyces aureofaciens, Streptomyces aureus, Streptomyces fungicidicus, Streptomyces griseochromogenes, Streptomyces griseus, Streptomyces lividans, Streptomyces olivogriseus, Streptomyces rameus, Streptomyces tanashiensis, Streptomyces vinaceus and Zymomonas mobilis.

Introduction of the recombinant DNA can be carried out by any of the methods for introducing DNA into the above host cells, for example, the method using calcium ion [Proc. Natl. Acad. Sci. USA, 69, 2110 (1972)], the protoplast method (Japanese Published Unexamined Patent Application No. 248394/88) and electroporation [Nucleic Acids Res., 16, 6127 (1988)].

When yeast is used as the host cell, YEp13 (ATCC 37115), YEp24 (ATCC 37051), YCp50 (ATCC 37419), pHS19, pHS15, etc. can be used as the expression vector.

As the promoter, any promoters capable of functioning in yeast can be used. Suitable promoters include PHO5 promoter, PGK promoter, GAP promoter, ADH promoter, gal 1 promoter, gal 10 promoter, heat shock polypeptide promoter, MFα1 promoter and CUP 1 promoter.

Examples of yeasts are those belonging to the genera Saccharomyces, Schizosaccharomyces, Kluyveromyces, Trichosporon, Schwanniomyces, Pichia and Candida, specifically, Saccharomyces cerevisiae, Schizosaccharomyces pombe, Kluyveromyces lactis, Trichosporon pullulans, Schwanniomyces alluvius, Pichia pastoris and Candida utilis.

Introduction of the recombinant DNA can be carried out by any of the methods for introducing DNA into yeast, for example, electroporation [Methods Enzymol., 194, 182 (1990)], the spheroplast method [Proc. Natl. Acad. Sci. USA, 81, 4889 (1984)] and the lithium acetate method [J. Bacteriol., 153, 163 (1983)].

When an animal cell is used as the host cell, pcDNAI, pcDM8 (commercially available from Funakoshi Co., Ltd.), pAGE107 (Japanese Published Unexamined Patent Application No. 22979/91), pAS3-3 (Japanese Published Unexamined Patent Application No. 227075/90), pCDM8 [Nature, 329, 840 (1987)], pcDNAI/Amp (Invitrogen Corp.), pREP4 (Invitrogen Corp.), pAGE103 [J. Biochem., 101, 1307 (1987)], pAGE210, pAMo, pAMoA, etc. can be used as the expression vector.

As the promoter, any promoters capable of functioning in animal cells can be used. Suitable promoters include the promoter of IE (immediate early) gene of cytomegalovirus (CMV), SV40 early promoter, metallothionein promoter, the promoter of a retrovirus, heat shock promoter, SRα promoter, etc. The enhancer of IE gene of human CMV may be used in combination with the promoter.

Examples of suitable animal cells are mouse myeloma cells, rat myeloma cells, mouse hybridomas, human-derived Namalwa cells and Namalwa KJM-1 cells, human embryonic kidney cells, human leukemia cells, African green monkey kidney cells, Chinese hamster-derived CHO cells, and HBT5637 (Japanese Published Unexamined Patent Application No. 299/88).

The mouse myeloma cells include SP2/0 and NSO; the rat myeloma cells include YB2/0; the human embryonic kidney cells include HEK293 (ATCC CRL-1573); the human leukemia cells include BALL-1; and the African green monkey kidney cells include COS-1 and COS-7.

Introduction of the recombinant DNA can be carried out by any of the methods for introducing DNA into animal cells, for example, electroporation [Cytotechnology, 3, 133 (1990)], the calcium phosphate method (Japanese Published Unexamined Patent Application No. 227075/90), lipofection [Proc. Natl. Acad. Sci. USA, 84, 7413 (1987)], and the method described in Virology, 52, 456 (1973).

When an insect cell is used as the host cell, the protein can be produced by using the methods described in Baculovirus Expression Vectors, A Laboratory Manual, W. H. Freeman and Company, New York (1992); Current Protocols in Molecular Biology; Molecular Biology, A Laboratory Manual; Bio/Technology, 6, 47 (1988), etc.

That is, the recombinant gene transfer vector and a baculovirus are cotransfected into insect cells to obtain a recombinant virus in the culture supernatant of the insect cells, and then insect cells are infected with the recombinant virus, whereby the protein can be produced.

The gene transfer vectors useful in this method include pVL1392, pVL1393 and pBlueBacIII (products of Invitrogen Corp.).

An example of the baculovirus is Autographa californica nuclear polyhedrosis virus, which is a virus infecting insects belonging to the family Barathra.

Examples of the insect cells are ovarian cells of Spodoptera frugiperda, ovarian cells of Trichoplusia ni, and cultured cells derived from silkworm ovary.

The ovarian cells of Spodoptera frugiperda include Sf9 and Sf21 (Baculovirus Expression Vectors, A Laboratory Manual); the ovarian cells of Trichoplusia ni include High 5 and BTI-TN-5Bl-4 (Invitrogen Corp.); and the cultured cells derived from silkworm ovary include Bombyx mori N4.

Cotransfection of the above recombinant gene transfer vector and the above baculovirus into insect cells for the preparation of the recombinant virus can be carried out by the calcium phosphate method (Japanese Published Unexamined Patent Application No. 227075/90), lipofection [Proc. Natl. Acad. Sci. USA, 84, 7413 (1987)], etc.

When a plant cell is used as the host cell, Ti plasmid, tobacco mosaic virus vector, etc. can be used as the expression vector.

As the promoter, any promoters capable of functioning in plant cells can be used. Suitable promoters include 35S promoter of cauliflower mosaic virus (CaMV), rice actin 1 promoter, etc.

Examples of suitable plant cells are cells of tobacco, potato, tomato, carrot, soybean, rape, alfalfa, rice, wheat and barley.

Introduction of the recombinant vector can be carried out by any of the methods for introducing DNA into plant cells, for example, the method using Agrobacterium (Japanese Published Unexamined Patent Application Nos. 140885/84 and 70080/85, WO94/00977), electroporation (Japanese Published Unexamined Patent Application No. 251887/85) and the method using particle gun (gene gun) (Japanese Patent Nos. 2606856 and 2517813).

### 6. Process for Producing the Protein of the Present Invention

The protein of the present invention can be produced by culturing the transformant obtained by the method of the above 5 in a medium, allowing the protein of the present invention to form and accumulate in the culture, and recovering the protein from the culture.

The host of the above transformant for producing the protein of the present invention may be any bacterium, yeast, animal cell, insect cell, plant cell or the like, but is preferably a bacterium, more preferably a microorganism belonging to the genus Escherichia, and further preferably a microorganism belonging to Escherichia coli.

When the protein of the present invention is expressed using yeast, an animal cell, an insect cell or a plant cell, a glycosylated protein can be obtained.

Culturing of the above transformant in a medium can be carried out by conventional methods for culturing the host.

For the culturing of the transformant obtained by using a procaryote such as Escherichia coli or yeast as the host, any of natural media and synthetic media can be used insofar as it is a medium suitable for efficient culturing of the transformant which contains carbon sources, nitrogen sources, inorganic salts, etc. which can be assimilated by the host used.

As the carbon sources, any carbon sources that can be assimilated by the host can be used. Examples of suitable carbon sources include carbohydrates such as glucose, fructose, sucrose, molasses containing them, starch and starch hydrolyzate; organic acids such as acetic acid and propionic acid; and alcohols such as ethanol and propanol.

As the nitrogen sources, ammonia, ammonium salts of organic or inorganic acids such as ammonium chloride, ammonium sulfate, ammonium acetate and ammonium phosphate, and other nitrogen-containing compounds can be used as well as peptone, meat extract, yeast extract, corn steep liquor, casein hydrolyzate, soybean cake, soybean cake hydrolyzate, and various fermented microbial cells and digested products thereof.

Examples of the inorganic salts include potassium dihydrogenphosphate, dipotassium hydrogenphosphate, magnesium phosphate, magnesium sulfate, sodium chloride, ferrous sulfate, manganese sulfate, copper sulfate and calcium carbonate.

Culturing is usually carried out under aerobic conditions, for example, by shaking culture or submerged spinner culture under aeration. The culturing temperature is preferably 15 to 40° C., and the culturing period is usually 5 hours to 7 days. The pH is maintained at 3.0 to 9.0 during the culturing. The pH adjustment is carried out by using an organic or inorganic acid, an alkali solution, urea, calcium carbonate, ammonia, etc.

If necessary, antibiotics such as ampicillin and tetracycline may be added to the medium during the culturing.

When a microorganism transformed with an expression vector comprising an inducible promoter is cultured, an inducer may be added to the medium, if necessary. For example, in the case of a microorganism transformed with an expression vector comprising lac promoter, isopropyl-β-D-thiogalactopyranosideor the like may be added to the medium; and in the case of a microorganism transformed with an expression vector comprising trp promoter, indoleacrylic acid or the like may be added.

For the culturing of the transformant obtained by using an animal cell as the host cell, generally employed media such as RPMI1640 medium [J. Am. Med. Assoc., 199, 519 (1967)], Eagle's MEM [Science, 122, 501 (1952)], DMEM [Virology, 8, 396 (1959)] and 199 medium [Proc. Soc. Biol. Med., 73, 1 (1950)], media prepared by adding fetal calf serum or the like to these media, etc. can be used as the medium.

Culturing is usually carried out at pH 6 to 8 at 25 to 40° C. for 1 to 7 days in the presence of 5% CO₂.

If necessary, antibiotics such as kanamycin, penicillin and streptomycin may be added to the medium during the culturing.

For the culturing of the transformant obtained by using an insect cell as the host cell, generally employed media such as TNM-FH medium (PharMingen, Inc.), Sf-900 II SFM medium (Life Technologies, Inc.), ExCell 400 and ExCell 405 (JRH Biosciences, Inc.) and Grace's Insect Medium [Nature, 195, 788 (1962)] can be used as the medium.

Culturing is usually carried out at pH 6 to 7 at 25 to 30° C. for 1 to 5 days.

If necessary, antibiotics such as gentamicin may be added to the medium during the culturing.

The transformant obtained by using a plant cell as the host cell may be cultured in the form of cells as such or after differentiation into plant cells or plant organs. For the culturing of such transformant, generally employed media such as Murashige-Skoog (MS) medium and White medium, media prepared by adding phytohormones such as auxin and cytokinin to these media, etc. can be used as the medium.

Culturing is usually carried out at pH 5 to 9 at 20 to 40° C. for 3 to 60 days.

If necessary, antibiotics such as kanamycin and hygromycin may be added to the medium during the culturing.

The protein of the present invention may be produced by intracellular production by host cells, extracellular secretion by host cells or production on outer membranes by host cells. The structure of the protein to be produced may be altered according to the production method.

When the protein of the present invention is produced in host cells or on outer membranes of host cells, it is possible to force the protein to be secreted outside the host cells by applying the method of Paulson, et al. [J. Biol. Chem., 264, 17619 (1989)], the method of Lowe, et al. [Proc. Natl. Acad. Sci. USA, 86, 8227 (1989); Genes Develop., 4, 1288 (1990)], or the methods described in Japanese Published Unexamined Patent Application No. 336963/93, WO94/23021, etc.

That is, extracellular secretion of the protein of the present invention by host cells can be caused by producing it in the form of a protein in which a signal peptide is added upstream of a protein containing the active site of the protein of the present invention by the use of recombinant DNA techniques.

It is also possible to increase the protein production by utilizing a gene amplification system using a dihydrofolate reductase gene or the like according to the method described in Japanese Published Unexamined Patent Application No. 227075/90.

Further, the protein of the present invention can be produced using an animal having an introduced gene (non-human transgenic animal) or a plant having an introduced gene (transgenic plant) constructed by redifferentiation of animal or plant cells carrying the introduced gene.

When the transformant producing the protein of the present invention is an animal or plant, the protein can be produced by raising or culturing the animal or plant in a usual manner, allowing the protein to form and accumulate therein, and recovering the protein from the animal or plant.

Production of the protein of the present invention using an animal can be carried out, for example, by producing the protein in an animal constructed by introducing the gene according to known methods [Am. J. Clin. Nutr., 63, 639S (1996); Am. J. Clin. Nutr., 63, 627S (1996); Bio/Technology, 9, 830 (1991)].

In the case of an animal, the protein of the present invention can be produced, for example, by raising a non-human transgenic animal carrying the introduced DNA of the present invention, allowing the protein to form and accumulate in the animal, and recovering the protein from the animal. The places where the protein is formed and accumulated include milk (Japanese Published Unexamined Patent Application No. 309192/88), egg, etc. of the animal. As the promoter in this process, any promoters capable of functioning in an animal can be used. Preferred promoters include mammary gland cell-specific promoters such as a casein promoter, β casein promoter, β lactoglobulin promoter and whey acidic protein promoter.

Production of the protein of the present invention using a plant can be carried out, for example, by culturing a transgenic plant carrying the introduced DNA encoding the protein of the present invention according to known methods [Soshiki Baiyo (Tissue Culture), 20 (1994); Soshiki Baiyo, 21 (1995); Trends Biotechnol., 15, 45 (1997)], allowing the protein to form and accumulate in the plant, and recovering the protein from the plant.

The protein of the present invention produced by using the transformant producing the protein of the present invention can be isolated and purified by conventional methods for isolating and purifying enzymes.

For example, when the protein of the present invention is produced in a soluble form in cells, the cells are recovered by centrifugation after the completion of culturing and suspended in an aqueous buffer, followed by disruption using a sonicator, French press, Manton Gaulin homogenizer, Dynomill or the like to obtain a cell-free extract.

A purified protein preparation can be obtained by centrifuging the cell-free extract to obtain the supernatant and then subjecting the supernatant to ordinary means for isolating and purifying enzymes, e.g., extraction with a solvent, salting-out with ammonium sulfate, etc., desalting, precipitation with an organic solvent, anion exchange chromatography using resins such as diethylaminoethyl (DEAE)-Sepharose, cation exchange chromatography using resins such as Q-Sepharose FF (Amersham Biosciences), hydrophobic chromatography using resins such as butyl Sepharose and phenyl Sepharose, gel filtration using a molecular sieve, affinity chromatography, chromatofocusing, and electrophoresis such as isoelectric focusing, alone or in combination.

When the protein is produced as an inclusion body in cells, the cells are similarly recovered and disrupted, followed by centrifugation to obtain a precipitate fraction. After the protein is recovered from the precipitate fraction by an ordinary method, the inclusion body of the protein is solubilized with a protein-denaturing agent.

The solubilized protein solution is diluted with or dialyzed against a solution containing no protein-denaturing agent or a solution containing the protein-denaturing agent at such a low concentration that denaturation of protein is not caused, whereby the protein is renatured to have normal higher-order structure. Then, a purified protein preparation can be obtained by the same isolation and purification steps as described above.

When the protein of the present invention or its derivative such as a glycosylated form is extracellularly secreted, the protein or its derivative such as a glycosylated form can be recovered in the culture supernatant.

That is, the culture is treated by the same means as above, e.g., centrifugation, to obtain a soluble fraction. A purified protein preparation can be obtained from the soluble fraction by using the same isolation and purification methods as described above.

An example of the protein obtained in the above manner is a protein consisting of the amino acid sequence of SEQ ID NO: 1.

It is also possible to produce the protein of the present invention as a fusion protein with another protein and to purify it by affinity chromatography using a substance having affinity for the fused protein. For example, the protein of the present invention can be produced as a fusion protein with protein A and can be purified by affinity chromatography using immunoglobulin G according to the method of Lowe, et al. [Proc. Natl. Acad. Sci. USA, 86, 8227 (1989); Genes Develop., 4, 1288 (1990)] and the methods described in Japanese Published Unexamined Patent Application No. 336963/93 and WO94/23021.

The protein of the present invention can also be produced as a fusion protein with a Flag peptide and purified by affinity chromatography using an anti-Flag antibody [Proc. Natl. Acad. Sci. USA, 86, 8227 (1989); Genes Develop., 4, 1288 (1990)], or can be produced as a fusion protein with polyhistidine and purified by affinity chromatography using a metal coordination resin having a high affinity for polyhistidine. Further, the protein can be purified by affinity chromatography using an antibody against the protein itself.

The protein of the present invention can also be produced by chemical synthetic methods such as the Fmoc method (the fluorenylmethyloxycarbonyl method) and the tBoc method (the t-butyloxycarbonyl method) based on the amino acid sequence information on the protein obtained above. Further, the protein can be chemically synthesized by using peptide synthesizers from Advanced ChemTech, Perkin-Elmer, Pharmacia, Protein Technology Instrument, Synthecell-Vega, PerSeptive, Shimadzu Corporation, etc.

### 7. Process for Producing a Dipeptide of the Present Invention

A dipeptide can be produced by allowing a culture of the microorganism or the transformant of the above 3 or a treated matter of the culture, or the protein of the present invention of the above 1, and one or more kinds of amino acids to be present in an aqueous medium, allowing the dipeptide to form and accumulate in the medium, and recovering the dipeptide from the medium.

### (1) Process for Producing a Dipeptide Using the Protein of the Present Invention as an Enzyme Source

When the protein of the present invention is used as an enzyme source in the production process of the present invention, one or more kinds, preferably one or two kinds of amino acids used as substrates may be any amino acids, preferably amino acids selected from the group consisting of L-amino acids, Gly and β-alanine (β-Ala), which can be used in any combination. Examples of L-amino acids are L-Ala, L-Gln, L-Glu, L-Val, L-Leu, L-Ile, L-Pro, L-Phe, L-Trp, L-Met, L-Ser, L-Thr, L-Cys, L-Asn, L-Tyr, L-Lys, L-Arg, L-His, L-Asp, L-α-aminobutylate (L-α-AB), L-azaserine, L-theanine, L-4-hydroxyproline (L-4-HYP), L-3-hydroxyproline (L-3-HYP), L-ornithine (L-Orn), L-citrulline (L-Cit) and L-6-diazo-5-oxo-norleucine.

The amino acids which are more preferably used in the above production process are one or two kinds of amino acids selected from the group consisting of L-Ala, L-Gln, L-Glu, Gly, L-Val, L-Leu, L-Ile, L-Pro, L-Phe, L-Trp, L-Met, L-Ser, L-Thr, L-Cys, L-Asn, L-Tyr, L-Lys, L-Arg, L-His, L-Asp and β-Ala. Further preferred amino acids are: a combination of L-Ala and one kind of amino acid selected from the group consisting of L-Phe, L-Trp, L-Met, L-Thr, L-Asn, L-Leu and L-Ile; a combination of L-Gln and one kind of amino acid selected from the group consisting of L-Met and L-Leu; a combination of L-Glu and L-Met; a combination of Gly and one kind of amino acid selected from the group consisting of L-Met, L-Thr and L-Leu; a combination of L-Val and one kind of amino acid selected from the group consisting of L-Met and L-Ser; a combination of L-Leu and one kind of amino acid selected from the group consisting of L-Pro, L-Phe, L-Met, L-Ser, L-Thr, L-Cys, L-Asn and L-Tyr; a combination of L-Ile and one kind of amino acid selected from the group consisting of L-Met and L-Ser; a combination of L-Pro and L-Met; a combination of L-Phe and one kind of amino acid selected from the group consisting of L-Trp and L-Met; a combination of L-Trp and one kind of amino acid selected from the group consisting of L-Trp and L-Met; a combination of L-Met and one kind of amino acid selected from the group consisting of L-Met, L-Ser, L-Thr, L-Cys, L-Asn, L-Tyr, L-Lys, L-Arg, L-His and L-Asp; a combination of L-Ser and one kind of amino acid selected from the group consisting of L-Ser, L-Thr and L-Asn; more preferably, a combination of L-Met and one kind of amino acid selected from the group consisting of L-Ala, Gly and L-Cys; and a combination of L-Leu and one kind of amino acid selected from the group consisting of L-Ala and Gly.

In the above process, the protein of the present invention is added in an amount of 0.01 to 100 mg, preferably 0.1 to 10 mg per mg of amino acid used as a substrate.

In the above process, the amino acid used as a substrate is added to the aqueous medium at the start or in the course of reaction to give a concentration of 0.1 to 500 g/L, preferably 0.2 to 200 g/L.

In the above process, ATP can be used as an energy source and is preferably used at a concentration of 0.5 mmol/L to 10 mol/L. ATP can be supplied in the form of a powder or a solution to an aqueous medium in which a dipeptide is formed and accumulated. ATP can also be supplied by imparting to the aqueous medium ATP-regenerating activity (activity to convert ADP formed in the dipeptide-forming process into ATP) utilizing glycolysis, polyphosphate kinase or the like. Specifically, ATP-regenerating activity can be imparted to the medium by adding culture cells of Corynebacterium ammoniagenes and a carbon source [Biosci. Biotechnol. Biochem., 65, 644 (2001)], adding polyphosphate kinase and polyphosphoric acid [J. Biosci. Bioeng., 91, 557 (2001)], and the like.

The aqueous medium used in the above process may comprise any components and may have any composition so far as the dipeptide-forming reaction is not inhibited. Suitable aqueous media include water and buffers such as phosphate buffer, carbonate buffer, acetate buffer, borate buffer, citrate buffer and Tris buffer. The aqueous medium may comprise alcohols such as methanol and ethanol, esters such as ethyl acetate, ketones such as acetone, and amides such as acetamide.

The dipeptide-forming reaction is carried out in the aqueous medium at pH 5 to 11, preferably pH 6 to 10, at 20 to 50° C., preferably 25 to 45° C., for 2 to 150 hours, preferably 6 to 120 hours.

The dipeptides produced by the above process include dipeptides in which amino acids, preferably amino acids selected from the group consisting of L-amino acids, Gly and β-Ala, more preferably amino acids selected from the group consisting of L-Ala, L-Gln, L-Glu, L-Val, L-Leu, L-Ile, L-Pro, L-Phe, L-Trp, L-Met, L-Ser, L-Thr, L-Cys, L-Asn, L-Tyr, L-Lys, L-Arg, L-His, L-Asp, L-α-AB, β-Ala, L-Azaserine, L-theanine, L-4-HYP, L-3-HYP, L-Orn, L-Cit, L-6-diazo-5-oxo-norleucine, Gly and β-Ala are linked with each other by a peptide bond. Further preferred are dipeptides in which two amino acids are linked by a peptide bond represented by formula (I):

R¹−R² (I)

(wherein when R¹ is L-Ala, R² is an amino acid selected from the group consisting of L-Leu, L-Ile, L-Phe, L-Trp, L-Met, L-Thr and L-Asn; when R¹ is L-Gln, R² is L-Leu or L-Met; when R¹ is L-Glu, R² is L-Met; when R¹ is Gly, R² is an amino acid selected from the group consisting of L-Leu, L-Met and L-Thr; when R¹ is L-Val, R² is L-Met or L-Ser; when R¹ is L-Leu, R² is an amino acid selected from the group consisting of L-Pro, L-Phe, L-Met, L-Ser, L-Thr, L-Cys, L-Asn, L-Tyr, L-Ala, L-Gln and Gly; when R¹ is L-Ile, R² is an amino acid selected from the group consisting of L-Met, L-Ser and L-Ala; when R¹ is L-Pro, R² is L-Met or L-Leu; when R¹ is L-Phe, R² is an amino acid selected from the group consisting of L-Trp, L-Met, L-Ala and L-Leu; when R¹ is L-Trp, R² is an amino acid selected from the group consisting of L-Trp, L-Met, L-Ala and L-Phe; when R¹ is L-Met, R² is an amino acid selected from the group consisting of L-Met, L-Ser, L-Thr, L-Cys, L-Asn, L-Tyr, L-Lys, L-Arg, L-His, L-Asp, L-Ala, L-Gln, L-Glu, Gly, L-Val, L-Leu, L-Ile, L-Pro, L-Phe and L-Trp; when R¹ is L-Ser, R² is an amino acid selected from the group consisting of L-Met, L-Ser, L-Thr, L-Asn, L-Val, L-Leu and L-Ile; when R¹ is L-Thr, R² is an amino acid selected from the group consisting of L-Ala, Gly, L-Leu, L-Met and L-Ser; when R¹ is L-Cys, R² is L-Leu or L-Met; when R¹ is L-Asn, R² is an amino acid selected from the group consisting of L-Ala, L-Leu, L-Met and L-Ser; when R¹ is L-Tyr, R² is L-Leu or L-Met; when R¹ is L-Lys, R² is L-Met; when R¹ is L-Arg, R² is L-Met; when R¹ is L-His, R² is L-Met; and when R¹ is L-Asp, R² is L-Met). Particularly preferred are dipeptides in which two amino acids are linked by a peptide bond represented by formula (I) (wherein when R¹ is L-Met, R² is an amino acid selected from the group consisting of L-Ala, Gly and L-Cys; and when R¹ is L-Leu, R² is an amino acid selected from the group consisting of L-Ala and Gly).

### (2) Process for Producing a Dipeptide Using a Culture of a Microorganism or a Transformant or a Treated Culture as an Enzyme Source

Examples of cultures of a microorganism or a transformant used as an enzyme source in the process of the present invention are cultures obtained by culturing the microorganism or transformant by the method of the above 6. Examples of the treated culture of the microorganism or transformant include treated cultures containing living cells having the same function as the culture as an enzyme source which is selected from the group consisting of concentrated culture, dried culture, cells obtained by centrifuging or filtering the culture, dried cells, freeze-dried cells, surfactant treated cells, solvent treated cells, enzyme treated cells and immobilized cells, and include ultrasonicated cells, mechanical-fricted cells, and crude enzyme extracts obtained from such treated cells.

When a culture of a transformant or a microorganism or a treated culture is used as an enzyme source, one or more kinds of amino acids used as substrates include the same amino acids as in the above (1).

The amount of the enzyme source to be added varies according to its specific activity, etc., but is, for example, 5 to 1000 mg (wet cell weight), preferably 10 to 400 mg per mg of amino acid used as a substrate.

The amino acid used as a substrate can be added to an aqueous medium in the same manner as in the above (1). ATP can be used as an energy source by allowing ATP to be present in an aqueous medium in the same manner as in the above (1). ATP can be supplied in the form of a powder or a solution to an aqueous medium in which a dipeptide is formed and accumulated. ATP can also be supplied by imparting to the aqueous medium ATP-regenerating activity (activity to convert ADP formed in the dipeptide-forming process into ATP) utilizing glycolysis, polyphosphate kinase or the like. Specifically, ATP-regenerating activity can be imparted to the medium by adding culture cells of Corynebacterium ammoniagenes and a carbon source [Biosci. Biotechnol. Biochem., 65, 644 (2001)], adding polyphosphate kinase and polyphosphoric acid [J. Biosci. Bioeng., 91, 557 (2001)], and the like.

As the aqueous medium, the media described in the above (1) can be used. In addition, a supernatant of the culture of a microorganism or a transformant used as an enzyme source can also be used as the aqueous medium.

The conditions for the dipeptide-forming reaction are the same as those in the above (1).

Examples of the dipeptides produced by the above process are the same dipeptides as in the above (1).

In the processes described in the above (1) and (2), recovery of the dipeptide formed and accumulated in the aqueous medium can be carried out by ordinary methods using active carbon, ion-exchange resins, etc. or by means such as extraction with an organic solvent, crystallization, thin layer chromatography and high performance liquid chromatography.

Certain embodiments of the present invention are illustrated in the following examples. These examples are not to be construed as limiting the scope of the invention.

### Example 1

### Construction of a Strain Expressing a Protein Having Dipeptide-Synthesizing Activity

Based on the nucleotide sequence information of the BL00235 gene encoding a protein of unknown function which has the nucleotide sequence of SEQ ID NO: 2 existing on the chromosomal DNA of Bacillus licheniformis ATCC 14580 (http://gib.genes.nig.ac.jp/single/index.php?spid=Blic_DSM 13_NOVOZYMES, http://www.ncbi.nlm.nih.gov/entrez/viewer.fcgi?val=5616098 4&itemID=4022&view=gbwithparts), a gene corresponding to the BL00235 gene (hereinafter merely referred to as BL00235 gene) was obtained from the chromosomal DNA of Bacillus licheniformis ATCC 14580 in the following manner.

First, Bacillus licheniformis ATCC 14580 was spread on YPGA medium [7 g/l yeast extract (Difco), 7 g/l Bacto-peptone (Difco), 7 g/l glucose and 1.5 g/l agar] and subjected to stationary culture overnight at 30° C. One platinum loop of grown cells was inoculated into 3 ml of YPG medium [7 g/l yeast extract (Difco), 7 g/l Bacto-peptone (Difco) and 7 g/l glucose], followed by shaking culture at 30° C. for 24 hours. The cells were collected by centrifugation, and the chromosomal DNA was prepared from the cells using Dneasy Kit (Qiagen, Inc.).

DNAs having the nucleotide sequences of SEQ ID NOS: 3 and 4 (hereinafter referred to as primer A and primer B, respectively) were synthesized by using a DNA synthesizer (Model 8905, PerSeptive Biosystems, Inc.). Primer A has a nucleotide sequence wherein a sequence containing the NcoI recognition sequence is added to the 5′ end of a region containing the initiation codon of the BL00235 gene on the chromosomal DNA of Bacillus licheniformis ATCC 14580. Primer B has a nucleotide sequence wherein a sequence containing the BamHI recognition sequence is added to the 5′ end of a nucleotide sequence complementary to a DNA sequence containing the N terminal amino acid sequence of the BL00235 gene.

PCR was carried out for amplification of a BL00235 gene fragment using the above primer A and primer B and the chromosomal DNA of Bacillus licheniformis ATCC 14580 as a template. PCR was carried out using 50 μL of a reaction mixture comprising 0.1 μg of the entire DNA, 0.5 μmol/l each of the primers, 2 units of KOD plus DNA polymerase (Toyobo Co., Ltd.), 5 μL of buffer for KOD plus DNA polymerase (10×) (Toyobo Co., Ltd.) and 200 μt mol/l each of dNTPs (dATP, dGTP, dCTP and dTTP) under the following conditions: incubation at 95° C. for 135 seconds; 30 cycles of 95° C. for 30 seconds, 52° C. for 45 seconds and 68° C. for 90 seconds; and a final incubation at 68° C. for 3 minutes.

One-tenth of the resulting reaction mixture was subjected to agarose gel electrophoresis to confirm that a ca. 1.3 kb DNA fragment containing the BL00235 gene was amplified by the PCR. Then, the DNA fragment was purified from the remaining reaction mixture using GFX-PCR and Gel Band purification kit (Amersham) and dissolved in 20 μl of TE.

The nucleotide sequence of the DNA was determined by a known method, whereby it was confirmed that the DNA has the nucleotide sequence of SEQ ID NO: 2 encoding the amino acid sequence of SEQ ID NO: 1.

The above-obtained DNA solution (5 μl) was subjected to reaction to cleave the DNA with restriction enzymes NcoI and BamHI. DNA fragments were separated by agarose gel electrophoresis, and a ca. 1.3 kb DNA fragment containing the BL00235 gene was recovered using GFX-PCR and Gel Band purification kit.

Expression vector pET-21d(+) (Novagen, Inc.) (0.2 μg) was cleaved with restriction enzymes NcoI and BamHI. DNA fragments were separated by agarose gel electrophoresis, and a ca. 5.4 kb DNA fragment was recovered in the same manner as above.

The above-obtained ca. 1.3 kb DNA fragment containing the BL00235 gene and the ca. 5.4 kb DNA fragment of expression vector pET-21d(+) obtained above were subjected to ligation reaction using a ligation kit (Takara Bio Inc.) at 16° C. for 16 hours.

Escherichia coli DH5α (Takara Bio Inc.) was transformed using the reaction mixture according to the method using calcium ion [Proc. Natl. Acad. Sci. USA, 69, 2110 (1972)], spread on LB agar medium containing 50 μg/ml ampicillin, and cultured overnight at 30° C.

A plasmid was extracted from a colony of the transformant that grew on the medium according to a known method, and the structure of the plasmid was analyzed using restriction enzymes. As a result, it was confirmed that an expression vector (pBL00235) in which the BL00235 gene having His-tag added to the N terminus was ligated downstream of the T7 promoter was obtained (FIG. 1).

Escherichia coli BL21(DE3) (Novagen, Inc.) was transformed using pBL00235 according to the method using calcium ion, spread on LB agar medium containing 50 μg/ml ampicillin, and cultured overnight at 30° C.

A plasmid was extracted from a colony of the transformant that grew on the medium according to a known method, and the structure of the plasmid was analyzed using restriction enzymes, whereby it was confirmed that the plasmid carried pBL00235.

### Example 2

### Production of a Protein Having Dipeptide-Synthesizing Activity

Escherichia coli BL21(DE3) carrying pBL00235 (Escherichia coli BL21(DE3)/pBL00235) obtained in Example 1 was inoculated into 3 ml of LB medium containing 50 μg/ml ampicillin in a test tube, and subjected to shaking culture at 37° C. for 6 hours. A portion of the resulting culture (100 μl) was inoculated into 100 mL of LB medium in a 500-ml Erlenmeyer flask and subjected to shaking culture at 37° C. for 3 hours. Then, isopropyl-β-D-thiogalactopyranoside (IPTG) was added to give a final concentration of 1 mmol/l, followed by further shaking culture at 28° C. for 15 hours. The resulting culture was centrifuged to obtain wet cells.

The wet cells were disrupted by ultrasonication and then centrifuged to obtain a supernatant. A His-tagged protein was purified from the obtained supernatant using HisTrap (His-tagged protein purification kit, Amersham).

### Example 3

### Production of Dipeptides Using the His-Tagged Protein

Reaction mixtures comprising the purified His-tagged protein obtained in Example 2 (0.5 mg/l), 50 mmol/l Tris-HCl buffer (pH 8.0), 12.5 mmol/1 magnesium sulfate, 12.5 mmol/l ATP, and respective combinations of L-amino acids and Gly shown in the first row and the leftmost column of Table 1 (12.5 mmol/l each) were prepared, and the resulting mixtures were subjected to reaction at 30° C. for 20 hours. After the completion of reactions, the amount of phosphoric acid liberated in the reaction mixtures was determined using Determiner LIP (Kyowa Medex Co., Ltd.) to confirm the progress of reactions. The reaction products were confirmed by the analysis with MALDI-TOFMS (Matrix Assisted Laser Desorption/Ionization—Time of Flight Mass Spectrometry).

The results are shown in Table 1

TABLE 1

| | Ala | Gln | Glu | Gly | Val | Leu | Ile | Pro |
|---|---|---|---|---|---|---|---|---|
| Ala | | | | | | AlaLeu or | ∘ | |
| | | | | | | LeuAla | | |
| Gln | | | | | | ∘ | | |
| Glu | | | | | | | | |
| Gly | | | | | | GlyLeu or | | |
| | | | | | | LeuGly | | |
| Val | | | | | | | | |
| Leu | | | | | | | | ∘ |
| Ile | | | | | | | | |
| Pro | | | | | | | | |
| Phe | | | | | | | | |
| Trp | | | | | | | | |
| Met | | | | | | | | |
| Ser | | | | | | | | |
| Thr | | | | | | | | |
| Cys | | | | | | | | |
| Asn | | | | | | | | |
| Tyr | | | | | | | | |
| Lys | | | | | | | | |
| Arg | | | | | | | | |
| His | | | | | | | | |
| Asp | | | | | | | | |
| β-Ala | | | | | | | | |

| | Phe | Trp | Met | Ser | Thr | Cys | Asn | Tyr |
|---|---|---|---|---|---|---|---|---|
| Ala | ∘ | ∘ | AlaMet or | | ∘ | | ∘ | |
| | | | MetAla, and | | | | | |
| | | | MetMet | | | | | |
| Gln | | | ∘ | | | | | |
| Glu | | | ∘ | | | | | |
| Gly | | | GlyMet or | | ∘ | | | |
| | | | MetGly, and | | | | | |
| | | | MetMet | | | | | |
| Val | | | ∘ | ∘ | | | | |
| Leu | ∘ | | LeuMet or | ∘ | ∘ | ∘ | ∘ | ∘ |
| | | | MetLeu, and | | | | | |
| | | | MetMet | | | | | |
| Ile | | | ∘ | ∘ | | | | |
| Pro | | | ∘ | | | | | |
| Phe | | ∘ | ∘ | | | | | |
| Trp | | ∘ | ∘ | | | | | |
| Met | | | MetMet | MetSer or | MetThr or | MetCys or | ∘ | ∘ |
| | | | | SerMet, and | ThrMet, and | CysMet, and | | |
| | | | | MetMet | MetMet | MetMet | | |
| Ser | | | | ∘ | ∘ | | ∘ | |
| Thr | | | | | | | | |
| Cys | | | | | | | | |
| Asn | | | | | | | | |
| Tyr | | | | | | | | |
| Lys | | | | | | | | |
| Arg | | | | | | | | |
| His | | | | | | | | |
| Asp | | | | | | | | |
| β-Ala | | | | | | | | |

| | Lys | Arg | His | Asp | B-Ala |
|---|---|---|---|---|---|
| Ala | | | | | |
| Gln | | | | | |
| Glu | | | | | |
| Gly | | | | | |
| Val | | | | | |
| Leu | | | | | |
| Ile | | | | | |
| Pro | | | | | |
| Phe | | | | | |
| Trp | | | | | |
| Met | ∘ | ∘ | ∘ | ∘ | |
| Ser | | | | | |
| Thr | | | | | |
| Cys | | | | | |
| Asn | | | | | |
| Tyr | | | | | |
| Lys | | | | | |
| Arg | | | | | |
| His | | | | | |
| Asp | | | | | |
| β-Ala | | | | | |

In Table 1, ◯ indicates that the progress of dipeptide-forming reaction was confirmed from the amount of liberated phosphoric acid. The dipeptides shown in Table 1 are dipeptides identified by the molecular weight of the reaction product as measured by MALDI-TOFMS.

As shown in Table 1, it was revealed that the protein of the present invention has the activity to form various kinds of dipeptides by linking one or two kinds of amino acids by a peptide bond.

### Example 4

### Analysis of the Structure of Dipeptides

Reaction mixtures comprising the purified His-tagged protein obtained in Example 2 (0.5 mg/l), 50 mmol/l Tris-HCl buffer (pH 8.0), 12.5 mmol/l magnesium sulfate, 12.5 mmol/l ATP, and two kinds of L-amino acids or Gly shown in the first row and the leftmost column of Table 2 (12.5 mmol/l each) were prepared, and the resulting mixtures were subjected to reaction at 30° C. for 20 hours. After the completion of reactions, the dipeptides shown in Table 2 among the reaction products were subjected to NMR (Nuclear Magnetic Resonance) analysis to confirm their structures and to measure their amounts formed.

TABLE 2

| | Ala | Gly | Cys |
|---|---|---|---|
| Met | MetAla | MetGly | MetCys |
| | 5.5 mmol/l | 4.4 mmol/l | 2.6 mmol/l |
| Leu | LeuAla | LeuGly | |
| | 2.2 mmol/l | | |

The amount of each of the formed dipeptides shown in Table 2 which were subjected to NMR analysis (mmol/l) is shown below the name of the respective dipeptide. The blank cell indicates that the experiment was not carried out, and the blank below the name of a dipeptide indicates that the structure of the dipeptide was confirmed but the amount formed was not measured.

### INDUSTRIAL APPLICABILITY

In accordance with the present invention, various kinds of dipeptides can be produced efficiently.

### SEQUENCE LISTING FREE TEXT

### SEQ ID NO: 3—Description of Artificial Sequence: Synthetic DNA

SEQ ID NO: 4—Description of Artificial Sequence: Synthetic DNA

1. A protein according to any of the following [1] to [3]:
[1] a protein having the amino acid sequence of SEQ ID NO: 1;
[2] a protein consisting of an amino acid sequence wherein one or more amino acid residues are deleted, substituted or added in the amino acid sequence of SEQ ID NO: 1 and having dipeptide-synthesizing activity; and
[3] a protein consisting of an amino acid sequence which has 80% or more homology to the amino acid sequence of SEQ ID NO: 1 and having dipeptide-synthesizing activity.

2. A DNA according to any of the following [1] to [3]:
1. DNA encoding the protein according to claim 1;
2. DNA having the nucleotide sequence of SEQ ID NO: 2; and
3. DNA which hybridizes with DNA having a nucleotide sequence complementary to the nucleotide sequence of SEQ ID NO: 2 under stringent conditions and which encodes a protein having dipeptide-synthesizing activity.

3. A recombinant DNA comprising the DNA according to claim 2.

4. A transformant carrying the recombinant DNA according to claim 3.

5. The transformant according to claim 4, wherein the transformant is a transformant obtained by using a microorganism as a host.

6. The transformant according to claim 5, wherein the microorganism is a microorganism belonging to the genus Escherichia.

7. A process for producing the protein according to claim 1, which comprises culturing a microorganism having the ability to produce said protein in a medium, allowing the protein to form and accumulate in the culture, and recovering the protein from the culture.

8. The process according to claim 7, wherein the microorganism having the ability to produce the protein is a transformant carrying a recombinant DNA encoding said protein.

9. A process for producing a dipeptide which comprises allowing (i) a culture of a microorganism having the ability to produce the protein according to claim 1 or a treated culture thereof, or (ii) said protein, and one or more kinds of amino acids to be present in an aqueous medium, allowing the dipeptide to form and accumulate in the medium, and recovering the dipeptide from the medium.

10. The process according to claim 9, wherein the microorganism having the ability to produce said protein is a transformant carrying a recombinant DNA encoding said protein.


SEQUENCE LISTING (4 sequences)

SEQ ID NO: 1 - protein, 425 aa, Bacillus licheniformis ATCC 14580
LTKRNKNLAIICQNKHLPFIFEEAERLGLKVTFFYNSAEDFPGNLPAVERCVPLPLFEDEEAAMDVVRQTFVEFPFDGVMTLFEPALPFTAKAAEALNLPGLPFTTMENCRNKNKTRSILQQNGLNTPVFHEFHTLADLENRKLSYPLVVKPVNGFSSQGVVRVDDRKELEEAVRKVEAVNQRDLNRFVHGKTGIVAEQFIDGPEFAIETLSIQGNVHVLSIGYKGNSKGPFFEEGVYIAPAQLKEETRLAIVKEVTGAVSALGIHQGPAHTELRLDKDGTPYVIEVGARIGGSGVSHYIVKESTGINFMQLVLQNALKPLESSEFEGEIRPVRTAGNYIIPVQGSGTFEKIDGLEEVKQRQEVKRVFQFMRRGAKILPYPHFSGYPGFILTSHHSYEECEAFYRELDDELHIIYQNNLTGTIGG

SEQ ID NO: 2 - DNA, 1275 bp, Bacillus licheniformis ATCC 14580
ttgacgaaacgaaacaaaaacttggccatcatttgtcaaaataagcacctgccatttatttttgaagaagctgagcgtttaggattgaaggtcacgttcttttacaattcggccgaagatttcccgggcaatcttccggctgtggaacgctgtgtgccgctgccgttgtttgaagatgaagaagcggcgatggatgtcgtcaggcagacatttgtcgaatttccgtttgacggcgtgatgacactgtttgaaccggctttgcctttcacggcaaaagctgctgaagctttgaatctgccggggcttcctttcacaacaatggaaaactgccgcaataaaaataaaacccgcagcatacttcagcaaaacggattgaatacgccggtttttcacgagttccatacgctcgctgacctggaaaacaggaaactgtcgtatccattagtcgtcaagccggtcaacggcttctcaagccagggcgtcgtccgtgttgatgatcggaaggagcttgaggaggccgtccggaaagtcgaggccgtcaaccaaagggacctcaatcgattcgtgcacggcaaaacgggcatcgtagccgagcaatttatcgatgggccggaattcgcgattgaaacgctgtcgattcaaggaaacgtacatgttctttcgattggatacaaagggaacagcaaagggccgttttttgaagaaggggtctatattgctccggctcaattgaaagaagagacgcgccttgcgatcgtcaaggaagtgacgggcgccgtttcagcgctgggcattcaccaggggccggctcatacagagctgaggctggacaaggatggaacaccttatgttatcgaggtgggcgccagaatcggcggttcaggggtttcccattacatcgtcaaagagagcacaggcatcaactttatgcagcttgtcttgcaaaatgcattaaagccgctggagagcagcgagtttgaaggcgagatcaggcctgtaaggacagccggcaattatatcattcctgtacagggctccggcacttttgaaaaaatcgacggactggaagaagtcaaacagaggcaggaggttaagcgggtgtttcaatttatgagaagaggcgcaaagatcctcccgtacccgcacttttccggttatccggggtttatattaacaagccatcattcttatgaagaatgtgaagctttctaccgggagctggatgatgagcttcatatcatttatcaaaataatttgacgggtacaataggaggt

SEQ ID NO: 3 - DNA, 32 bp, Artificial Sequence
  Description of Artificial Sequence Synthetic DNA
cgagctcccatggcgaaacgaaacaaaaactt

SEQ ID NO: 4 - DNA, 28 bp, Artificial Sequence
  Description of Artificial Sequence Synthetic DNA
gcggatcccctcctattgtacccgtcaa